(19) United States
(12) Patent Application Publication
(10) Pub. No.: US 2019/0189269 A1
(43) Pub. Date: Jun. 20, 2019

(54) MEDICAL IMAGING APPARATUS AND METHOD FOR DISPLAYING MEDICAL IMAGES

(71) Applicant: CANON KABUSHIKI KAISHA, Tokyo (JP)

(72) Inventor: Kouichi Kato, Yokohama-shi (JP)

(21) Appl. No.: 16/215,243

(22) Filed: Dec. 10, 2018

(30) Foreign Application Priority Data
Dec. 15, 2017 (JP) 2017-240948

(51) Int. Cl. G16H 30/40; G06T 7/00; G06T 11/60; G06F 16/53

(52) U.S. Cl. CPC G16H 30/40; G06T 7/0014; G06T 11/60; G06F 16/53; G06T 2200/24; G06T 2207/30096; G06T 2207/10132; G06T 2207/20212; G06T 2207/10124; G06T 2207/30101

(57) ABSTRACT

A medical imaging apparatus includes a radiogram interpretation image acquisition unit that acquires a radiogram interpretation image superimposing an ultrasound image and a photoacoustic image so that imaging angles of view to a subject overlap with each other, a reference image acquisition unit that acquires a reference image to estimate a nature of the radiogram interpretation image, and a side-by-side display unit that displays the radiogram interpretation image and the reference image side by side. The reference image acquisition unit acquires the reference image superimposing a tumor image and a blood vessel image.

### BACKGROUND

### Field

The present disclosure relates to a medical imaging apparatus that displays a medical image acquired by a modality and a method for displaying the medical image.

### Description of the Related Art

Photoacoustic tomography (PAT) is used as a method for forming an image of blood vessels. PAT is a technique for irradiating a subject with a near-infrared ray and detecting a photoacoustic wave emitted from the subject by using an acoustic wave detector to form an image. An apparatus for implementing this technique is known as a photoacoustic apparatus. “Functional photoacoustic microscopy for high-resolution and noninvasive in vivo imaging” Zhang H. F., Maslov K., Stoica G., Wang L. V. (2006) Nature Biotechnology, 24(7), pp. 848-851 discusses a photoacoustic apparatus configured to noninvasively image a running state of blood vessels and oxygen saturation in a living body by selecting irradiation light in consideration of an absorption spectrum of hemoglobin.

In a medical image diagnosis by a doctor, an image undergoing radiogram interpretation is compared with a previously acquired reference image to investigate characteristics of a lesion area. The reference image includes information about a disease and a clinical condition. If similar characteristics to the reference image are found, the disease or the clinical condition can be diagnosed. U.S. Patent Application Publication No. 2016/0343132 discusses a method for estimating benignancy/malignancy of a tumor by using a radiogram interpretation target image. The radiogram interpretation target image is formed by superimposing, on an ultrasound echo image of a breast captured by an ultrasound apparatus, a photoacoustic image visualizing the running state of the blood vessels in a region suspected to have the tumor identified by the echo image. U.S. Patent Application Publication No. 2016/0343132 discusses a technique for displaying several examples of pre-acquired reference images according to their case classification and malignancy side by side on the same screen as the radiogram interpretation target image.

When a radiogram interpretation target image is formed by superimposing a photoacoustic image on an echo image as with the technique discussed in U.S. Patent Application Publication No. 2016/0343132, a comparison becomes difficult depending on a way of presenting the reference images. Accordingly, in order to reliably find a similar case, it may take too much time, and a more similar image may possibly be overlooked.

### SUMMARY

The present disclosure is directed to providing a medical imaging apparatus with which the comparison between the radiogram interpretation target image and the reference image becomes easy and to a method for displaying a medical image when the radiogram interpretation target image including a photoacoustic image and another modality image is interpreted.

According to an aspect of the present disclosure, a medical imaging apparatus includes a radiogram interpretation image acquisition unit configured to acquire a radiogram interpretation image superimposing an ultrasound image and a photoacoustic image so that imaging angles of view to a subject overlap with each other, a reference image acquisition unit configured to acquire a reference image to estimate a nature of the radiogram interpretation image, and a side-by-side display unit configured to display the radiogram interpretation image and the reference image side by side. The reference image acquisition unit acquires the reference image superimposing a tumor image and a blood vessel image.

According to another aspect of the present disclosure, a medical imaging apparatus includes a radiogram interpretation image acquisition unit configured to acquire a radiogram interpretation image superimposing an ultrasound image and a photoacoustic image so that imaging angles of view to a subject overlap with each other, a reference image acquisition unit configured to acquire a reference image to estimate a nature of the radiogram interpretation image, and a side-by-side display unit configured to display the radiogram interpretation image and the reference image side by side. The reference image acquisition unit acquires the reference image superimposing a previously acquired ultrasound image and a previously acquired photoacoustic image captured before the radiogram interpretation image is captured.

According to yet another aspect of the present disclosure, a method for displaying medical images includes acquiring a radiogram interpretation image superimposing an ultrasound image and a photoacoustic image so that imaging angles of view to a subject overlap with each other, acquiring a reference image to estimate a nature of the radiogram interpretation image, and displaying the radiogram interpretation image and the reference image side by side. In acquiring the reference image, the reference image superimposing a tumor image and a blood vessel image is acquired.

Further features will become apparent from the following description of exemplary embodiments with reference to the attached drawings.

### BRIEF DESCRIPTION OF THE DRAWINGS

FIGS. 1A, 1B, 1C, 1D, and 1E illustrate a display example (FIG. 1A), radiogram interpretation images (FIGS. 1B and 1C), and reference images before their superimposition (FIGS. 1D and 1E) according to a first exemplary embodiment.

FIG. 2 schematically illustrates a configuration of a medical imaging apparatus according to the first exemplary embodiment.

FIG. 3 is a flowchart illustrating a method for displaying a reference image according to the first exemplary embodiment.

FIGS. 4A and 4B illustrate display examples of reference images according to a reference form (FIG. 4A) and according to the first exemplary embodiment (FIG. 4B).

FIGS. 5A, 5B, and 5C illustrate display layouts of reference images according to a second exemplary embodiment.

FIG. 6 illustrates a method for adjusting a reference image and a radiogram interpretation image according to a third exemplary embodiment.

### DESCRIPTION OF THE EMBODIMENTS

Exemplary embodiments of the present disclosure will be described below with reference to the accompanying drawings. Sizes, materials, shapes, and relative arrangements of components as described below can be changed as required depending on a configuration of an apparatus according to the present disclosure and other various conditions and are not intended to limit the scope of the present disclosure to the following descriptions.

The present disclosure relates to a technique for processing subject information generated by photoacoustic imaging. The present disclosure can relate to a medical image processing apparatus and a method for controlling the medical image processing apparatus, or a method for acquiring a medical image, a signal processing method, and an image processing method. The present disclosure can also relate to a program to be executed by an information processing apparatus having a hardware resource such as a central processing unit (CPU), and as a storage medium storing the program. The subject information is acquired by an apparatus using the photoacoustic tomography technique. More specifically, the apparatus irradiates a subject with light (electromagnetic wave) and receives (detects) an acoustic wave generated at a specific position inside the subject or on the surface of the subject by the photoacoustic effect and is propagated. Radiogram interpretation information and reference information according to the present disclosure as parts of the subject information include a position of a generation source of the acoustic wave generated by light irradiation, an initial sound pressure inside the subject, an optical energy absorption density distribution and an absorption coefficient derived from an initial sound pressure distribution, and densities of substances constituting a subject tissue. More specifically, the densities of the substances constituting the subject tissue include a blood component physical property value such as an oxidization-reduction hemoglobin density and oxygen saturation calculated from the oxidization-reduction hemoglobin density, and densities of fat, collagen, and moisture. These values can be obtained as numerical data as well as distribution information associated with each position inside the subject. The above-described distribution information forms a radiogram interpretation image and a reference image.

An ultrasonic wave and a photoacoustic wave according to the present specification are included in an elastic wave called an acoustic wave. An acoustic wave generated by an electromechanical transducer is referred to as an ultrasonic wave or a transmitted wave, and an acoustic wave reflected or scattered by the subject and received by an electromechanical transducer is referred to as an ultrasonic wave, a received wave, or an echo wave. An acoustic wave generated by the photoacoustic effect is referred to as a photoacoustic wave.

A radiogram interpretation image according to the present specification includes a radiogram interpretation target image of a patient's target region specified by an inspection order and captured by using a modality to diagnose the patient. The radiogram interpretation image includes one or more of a photoacoustic image captured by a photoacoustic apparatus or an ultrasound image captured by an ultrasound apparatus. Each of the photoacoustic apparatus and the ultrasound apparatus can be configured to include the function of the other, and the apparatuses can also be implemented as a common modality.

A previously acquired image according to the present specification includes a photoacoustic image and an ultrasound image captured before the radiogram interpretation image is captured, and is an image stored in an image database in association with an annotation such as a diagnostic result and an image generation condition. The previously acquired image is accumulated in the image database in association with meta information related to image data so that a search is possible. Thus, the previously acquired image can indicate a set of related images.

A candidate image according to the present specification is an image that serves as a candidate for a reference image to be referenced by a radiogram interpreter for diagnosis. The candidate image becomes the reference image when a candidate image is specified by an operator of the medical imaging apparatus of the present disclosure. The candidate image refers to an image or an image group that is searched from among previously acquired images based on related information such as a keyword and is extracted as a candidate for the reference image. The candidate image can be prioritized by matching with the related information.

The operator of the medical imaging apparatus includes a radiogram interpretation doctor. The reference image refers to an image selected from among candidate images by the radiogram interpreter to be used in a comparison with a radiogram interpretation image to diagnose the radiogram interpretation image. In other words, the previously acquired image is an image used as the candidate image for the reference image to be compared with the radiogram interpretation image.

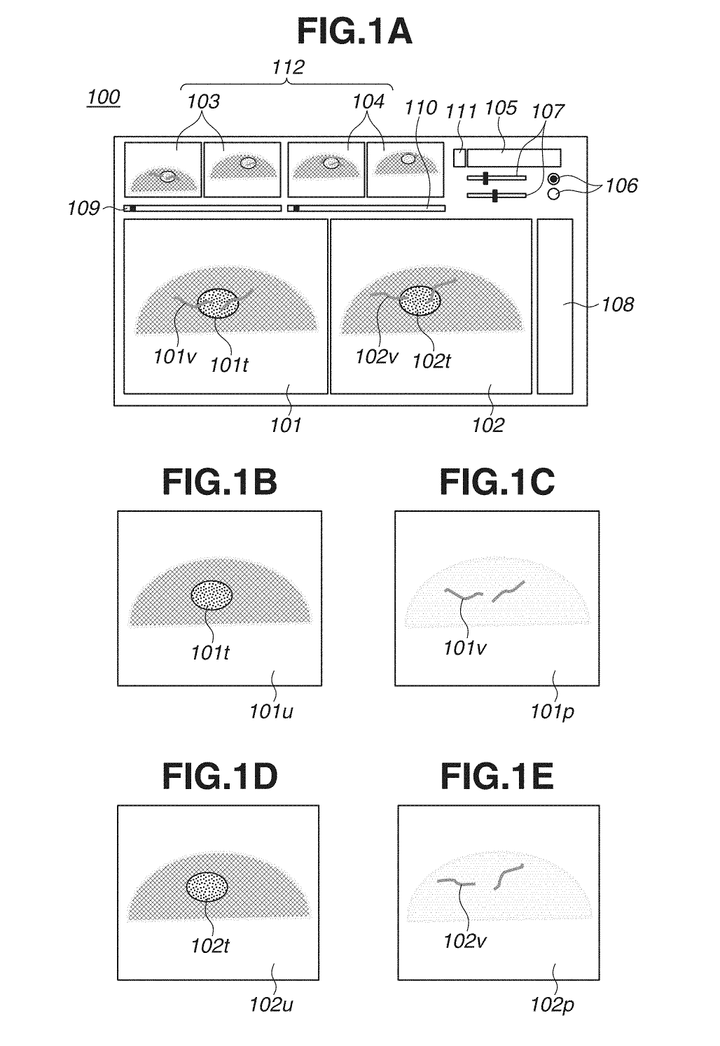

An example of comparative display of a radiogram interpretation image and a reference image performed by a medical imaging apparatus 200 according to a first exemplary embodiment will be described below with reference to FIGS. 1A to 1E.

### <Display Screen>

As illustrated in FIG. 1A, a display screen 100 displays a radiogram interpretation image 101, a reference image 102, reference image candidates 103 and 104, a keyword input portion 105, and a meta-information display portion 108 that displays diagnostic result data and image generation data of the radiogram interpretation image 101. The display screen 100 is a component of a display unit 218 (described below). The keyword input portion 105 is a component of an information input unit 212 (described below). While the radiogram interpretation image 101 and the reference image 102 are horizontally displayed side by side, the present disclosure includes other side-by-side display formats, such as, for example, vertical side-by-side arrangement, as modifications as long as the comparison can be easily performed in image diagnosis. In addition, each of the radiogram interpretation image 101 and the reference image 102 can also be displayed on two or more windows.

As illustrated in FIG. 1A, the display screen 100 according to the present exemplary embodiment includes setting buttons 106 and image adjustment sliders 107 for inputting an instruction for adjusting image quality of the reference image 102 or the radiogram interpretation image 101. The setting buttons 106 and the image adjustment sliders 107 are components of an adjustment value input unit 219 (described below).

### <Radiogram Interpretation Image>

The radiogram interpretation image 101 displays a superimposed image of an ultrasound image 101u and a photoacoustic image 101p. The ultrasound image 101u presents a tumor outer edge 101t illustrated in FIG. 1B, and the photoacoustic image 101p presents a blood vessel running state 101v based on a photoacoustic contrast illustrated in FIG. 1C. By using an image formed by superimposing the ultrasound image 101u and the photoacoustic image 101p in this way, the radiogram interpreter performs radiogram interpretation about a nature of the tumor, i.e., a grade of malignancy based on the tumor image 101t presenting the tumor outer edge and the blood vessel image 101v presenting the blood vessel running state.

### <Reference Image>

The reference image 102 according to the present exemplary embodiment is an image superimposing a tumor image 102t illustrated in FIG. 1D and a blood vessel image 102v illustrated in FIG. 1E. The tumor image 102t and the blood vessel image 102v are selected based on a previously acquired ultrasound image 102u and a previously acquired photoacoustic image 102p, respectively, captured before the radiogram interpretation image 101 is captured. In other words, the reference image 102 is formed by superimposing the previously acquired ultrasound image 102u and the previously acquired photoacoustic image 102p captured before the radiogram interpretation image 101 is captured. As described above, by using the reference image 102 superimposing the tumor image 102t and the blood vessel image 102v, the radiogram interpreter can easily compare the reference image 102 with the radiogram interpretation image 101 with which a nature of the tumor is to be evaluated. The tumor image 102t is associated with a nature of a tumor by a differential diagnosis. In other words, the tumor image 102t is associated with the grade of malignancy established by the differential diagnosis.

While, in the tumor image according to the present specification, a boundary between a tumor portion and a normal portion cannot be clearly determined by the photoacoustic imaging, the tumor image corresponds to a region having a boundary that can be determined by another imaging method or a biopsy. Thus, according to the present specification, the tumor includes a malignant tumor, a benign tumor, a cyst, a pustule, and inflammation, each having a different nature. As for a region suspected to have the above-described disease with the different tumor nature, the differential diagnosis may or may not have been established. The display screen 100 can display an image that does not superimpose the radiogram interpretation image 101 and the reference image 102. A radiogram interpretation image acquisition unit 205 and a reference image acquisition unit 213 (described below) can set transmittance of at least one of the photoacoustic image 101p (102p) and the ultrasound image 101u (102u) to 100% and set the transmittance of another image to a value lower than 100%. In other words, the radiogram interpretation image acquisition unit 205 and the reference image acquisition unit 213 can set the transmittance of at least one of the photoacoustic image 101p (102p) and the ultrasound image 101u (102u) to 0% and set the transmittance of another image to a value greater than 0%.

To change the reference image 102, by selecting either of the candidate images 103 and 104, the reference image 102 is replaced with either of the candidate images 103 and 104. The candidate images 103 and 104 are displayed based on a priority given to a previously acquired image group prioritized by the reference image acquisition unit 213 (described below).

### <Candidate Image>

The candidate image 103 is a group of appropriate images of disease examples that are identical or similar to the radiogram interpretation image 101. The candidate image 104 is a group of images estimated to be an image not appropriate to be differentiated and having a different nature from the radiogram interpretation image 101. The candidate images 103 and 104 are acquired from an image database 220 (described below) based on at least one of image information that is extracted based on the radiogram interpretation image 101 and related information about a case. The image information that is extracted based on the radiogram interpretation image 101 and the related information about a case will be described below with respect to an overall configuration of the medical imaging apparatus 200. The candidate images 103 and 104 are displayed as thumbnails each having a low display resolution and a small display size so as to be distinguished from the reference image 102 and in consideration of visibility of the screen.

In a display example of the display screen 100 according to the present exemplary embodiment, two higher-priority candidate images 103 and two higher-priority candidate images 104 are displayed, respectively. Lower-priority candidate images 103 and 104 can also be displayed by scrolling the screen using sliders 109 and 110. The keyword input portion 105 for giving the priority to the candidate image group includes an input portion 411 for specifying a keyword by which a desirable candidate image group is input.

Similar to the reference image 102, each of the candidate images 103 and 104 for the reference image 102 superimposes a tumor image and a blood vessel image. In other words, similar to the reference image 102, each of the candidate images 103 and 104 for the reference image 102 is also formed by superimposing a previously acquired ultrasound image and a previously acquired photoacoustic image captured before the radiogram interpretation image 101 is captured.

While cross sections (axial sections) are displayed in FIGS. 1A, 1B, 1C, 1D, and 1E, coronal planes (coronal sections), sagittal planes (sagittal sections), or arbitrary sections can be displayed. In addition to two-dimensional images, three-dimensional images and moving images can also be displayed on the display.

### <Overall Configuration of Medical Imaging Apparatus>

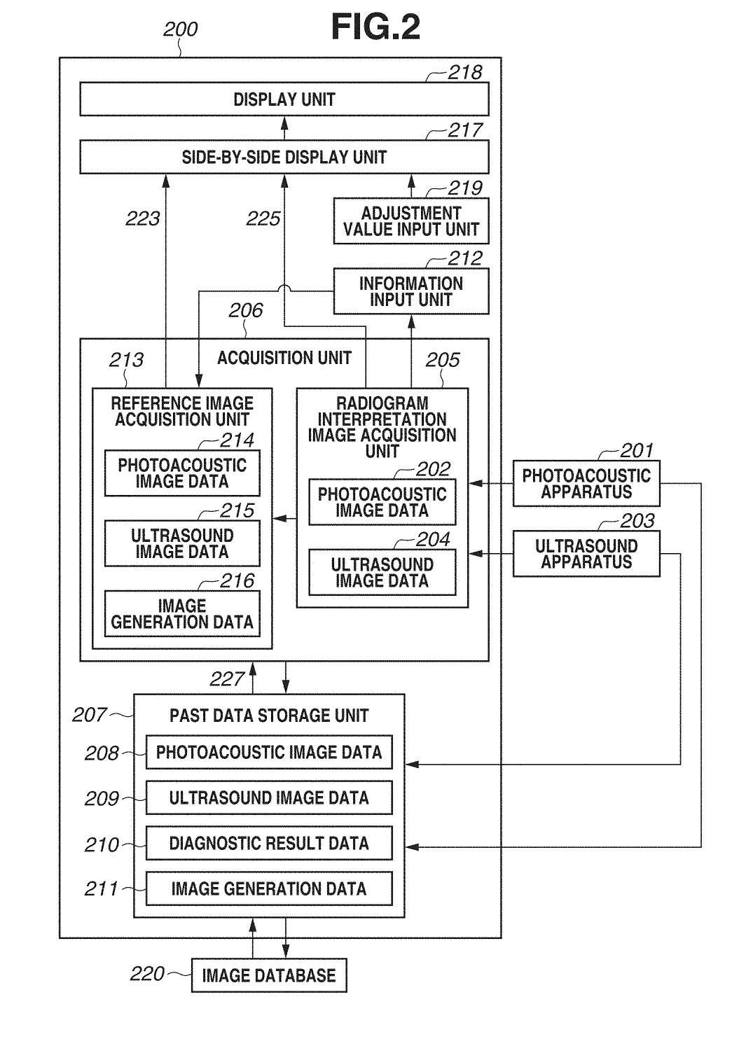

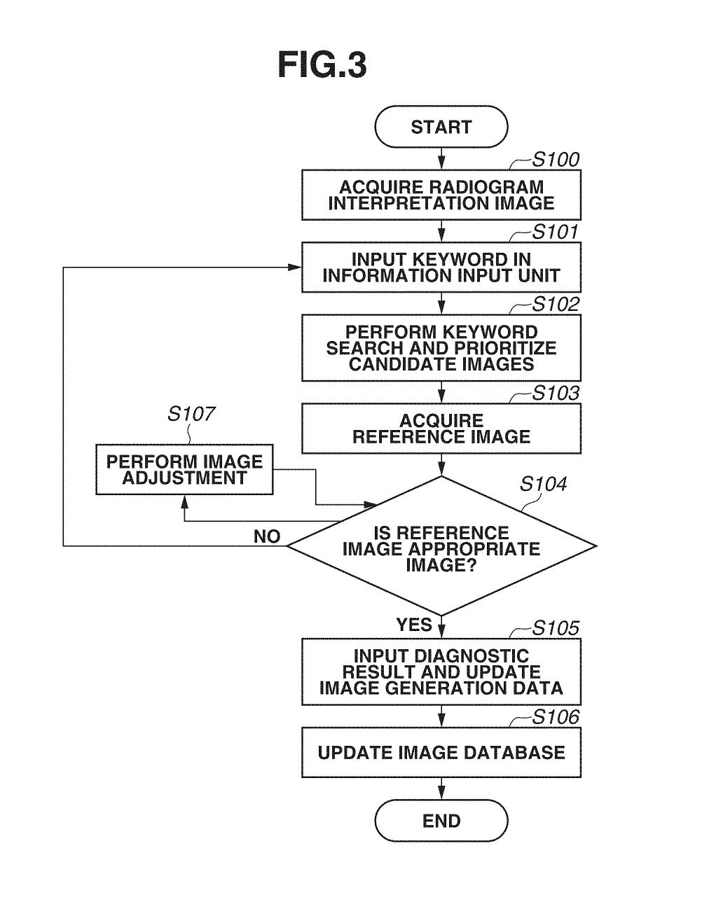

The configuration of the medical imaging apparatus 200 and a sequence of a method for displaying a medical image according to the present exemplary embodiment will be described below with reference to FIGS. 2 and 3, respectively. FIG. 2 is a block diagram schematically illustrating the configuration of the medical imaging apparatus 200. FIG. 3 is a flowchart illustrating the sequence of diagnosis on a radiogram interpretation image by using the radiogram interpretation image and the reference image.

### <Acquisition Unit>

An acquisition unit 206 includes a radiogram interpretation image acquisition unit 205 that acquires radiogram interpretation image data 225 from a photoacoustic apparatus 201 and an ultrasound apparatus 203, and a reference image acquisition unit 213 that acquires reference image data 227 from the image database 220 via a past data storage unit 207.

The radiogram interpretation image acquisition unit 205 and the reference image acquisition unit 213 are configured to acquire related information associated with image data together with the target image data. The radiogram interpretation image acquisition unit 205 and the reference image acquisition unit 213 can exchange the image data and the related information (related data) with another data output component via wired communication, wireless communication, and optical communication as well as via a recording medium and a storage device. The medical imaging apparatus 200 can be installed in a remote location from the photoacoustic apparatus 201 and the ultrasound apparatus 203. Alternatively, the photoacoustic apparatus 201 or the ultrasound apparatus 203 can be installed in a different location from the medical imaging apparatus 200.

When transferring the reference image data 227 from the image database 220 to the acquisition unit 206 via the past data storage unit 207, the diagnostic result data and the image generation data are collectively transferred with the image data.

### <Radiogram Interpretation Image Acquisition Unit>

The radiogram interpretation image acquisition unit 205 is a component that acquires the radiogram interpretation image data 225 related to a radiogram interpretation target medical image to be used for image diagnosis before a definite diagnosis is established, and that outputs the image data to a side-by-side display unit 217 (described below). The radiogram interpretation image acquisition unit 205 acquires photoacoustic image data 202 from the photoacoustic apparatus 201 and acquires ultrasound image data 204 from the ultrasound apparatus 203. The radiogram interpretation image acquisition unit 205 also performs processing for superimposing the photoacoustic image data 202 and the ultrasound image data 204 to generate the radiogram interpretation image 101 superimposing the photoacoustic image 101p and the ultrasound image 101u. In the present specification, an image acquisition process includes an image generation process.

The radiogram interpretation image acquisition unit 205 outputs, to the side-by-side display unit 217 (described below), the radiogram interpretation image data 225 for generating the radiogram interpretation image 101 including the superimposed images. In other words, if the radiogram interpretation image 101 includes a finding indicating a suspected tumor, the radiogram interpretation image acquisition unit 205 performs processing for superimposing the photoacoustic image data 202 and the ultrasound image data 204 to generate the radiogram interpretation image 101 superimposing the blood vessel image 101v and the tumor image 101t.

The photoacoustic image data 202 and the ultrasound image data 204 constituting the radiogram interpretation image data 225 are a combination in which coordinate systems thereof are associated with each other and their positions can be aligned. An example of the combination of the medical images that can be aligned is a combination of medical images capturing an identical region or overlapping regions of a subject. More specifically, since the coordinate systems of the photoacoustic image data 202 and the ultrasound image data 204 are associated with each other, the radiogram interpretation image 101 is formed by superimposing the photoacoustic image data 202 and the ultrasound image data 204 so that angles of view in imaging the subject overlap with each other.

As the radiogram interpretation image data 225, a data format of any dimension can be employed, such as volume data generated by three-dimensional tomography or tomographic image data generated by capturing a part of a tissue section. As for the radiogram interpretation image data 225, a medical image can include information about an annotation indicating a region of interest (ROI).

### <Past Data Storage Unit>

The past data storage unit 207 stores a medical image that previously underwent a diagnosis. The past data storage unit 207 stores photoacoustic image data 208, ultrasound image data 209, diagnostic result data 210, and image generation data 211. The past data storage unit 207 outputs the selected reference image data 227 to the reference image acquisition unit 213 (described below).

The past data storage unit 207 acquires the photoacoustic image data 208, the ultrasound image data 209, the diagnostic result data 210, and the image generation data 211 from the image database 220 and stores these data therein. According to the present exemplary embodiment, the image database 220 is located external to the medical imaging apparatus 200, but connected to the medical imaging apparatus 200 so that the data can be transmitted to and received from the medical imaging apparatus 200.

The diagnostic result data 210 refers to related information about a medical image obtained at the time of image diagnosis. The diagnostic result data 210 includes a disease name, clinical conditions, blood vessel characteristics, a lesion area, a lesion area size, a lesion area depth, a lesion location division, lesion boundary positional information, a ROI, a pathological result, clinical inspection data, gender, age, clinical history, treatment history, medication history, and background information of a patient. The diagnostic result data 210 also includes a degree of recommendation as a reference image based on an impression made on the radiogram interpreter, an experience value of the radiogram interpreter, a skill of the radiogram interpreter, a degree of diagnostic difficulty, and usefulness as a reference image. In addition, the radiogram interpreter can add an arbitrary criterion for selecting a reference image to the diagnostic result data 210.

When the radiogram interpreter determines that a reference image is useful for reference for a future radiogram interpreter as a typical case of a specific disease, the degree of recommendation is set to a high value as a reference image for the disease. The experience value of the radiogram interpreter is a score representing the number of times of registration in the image data and data of the related information stored in the past data storage unit 207. The skill of the radiogram interpreter is a score representing a hit rate in comparison between a previously acquired image diagnostic result and a diagnostic result of a subsequent definite diagnosis such as a pathological examination or is a score representing an experience level of the image diagnosis. The degree of diagnostic difficulty is a score representing a difference between the previously acquired image diagnostic result and the diagnostic result of the subsequent definite diagnosis such as the pathological examination. A reference image that has the difference therebetween is determined as having a high degree of diagnostic difficulty. The reference image that has the high degree of diagnostic difficulty enables the radiogram interpreter to know a point to be cautious in a diagnosis. Such a reference image is an example of a case that is likely to cause an incorrect diagnosis. The usefulness as a reference image is a score representing, for example, the number of times a reference image is used or how useful a reference image has been for the radiogram interpreter who referenced the reference image for a diagnosis.

The diagnostic result data 210 also includes data of a comment including a target point and an attention point in the image diagnosis for the future radiogram interpreter. The data is recorded as a text, a symbol, a moving image, and a sound. The diagnostic result data 210 includes information for directly specifying the target point in a reference image or a moving image (described below) in the screen by using an arrow. The image generation data 211 includes parameters used for adjusting image quality when displaying the radiogram interpretation image 101. The parameters include a luminance adjustment value, a contrast adjustment value, a target position and range within a reference image, an enlargement factor, a projected image generation parameter, and a superimposed image generation parameter. The projected image generation parameter includes a setting value related to a projected image type (such as the maximum value projection and the minimum value projection), a projection range, luminance, and contrast of the projected image. The superimposed image generation parameter includes information about an original image before superimposition, a superimposition ratio, a display setting such as coloring, and alignment information. The image generation data 211 is used both for superimposition of the reference image 102 and for superimposition of the radiogram interpretation image 101 based on a method for generating a superimposition image by an expert that is provided in a data form.

As described above, it is possible to manage a score of the related information including the diagnostic result data 210 and the image generation data 211 other than the photoacoustic image data 208 and the ultrasound image data 209 stored in the past data storage unit 207. In other words, the related information about the radiogram interpreter is parametric information that is parametrically managed. The parametric information includes a tumor size, a lesion area size, a blood vessel diameter, and a blood vessel wall thickness that are measured by the radiogram interpreter based on the radiogram interpretation image 101 as well as a standard deviation of a blood vessel running direction.

In the past data storage unit 207, the photoacoustic image data 208 and the ultrasound image data 209 are a combination in which coordinate systems are associated with each other and can be aligned, similar to those in the radiogram interpretation image acquisition unit 205. The data in the past data storage unit 207 can include volume data or tomographic image data (slice image data) and can also include information about the ROI. In the previously acquired image data, a photoacoustic image includes a blood vessel image and an oxygen saturation image.

In recent years, image data has included metadata and image data conforming to the Digital Imaging and Communications in Medicine (DICOM) standard. The metadata includes, for example, a patient name, age, birth date, gender, inspection, series, and information about an image. In the present disclosure, since a DICOM standard image can also be handled, a part of the diagnostic result data such as the age and the gender can be stored in the metadata of the photoacoustic image data 202 and the ultrasound image data 204. More specifically, it is not necessary to store the image data 202, 204, 208, and 209 and the data 210 and 211 in clearly different electronic data files.

The past data storage unit 207 supplies the diagnostic result data and the image generation data to the radiogram interpretation image acquisition unit 205, and the data are stored in the acquisition unit 206 when diagnosis is completed. Previously acquired image data acquired from a database different from the image database 220 can be transferred to the acquisition unit 206 to be stored therein.

The data in the past data storage unit 207 is a set of an image having undergone a previous diagnosis and related information. Both a previous radiogram interpretation image of a subject and an image of a different subject can be used.

### <Reference Image Acquisition Unit>

To estimate the nature of the radiogram interpretation image 101, the reference image acquisition unit 213 acquires a reference image estimated to have a high correlation with the radiogram interpretation image 101 from among a group of medical images associated with a diagnostic result of a definite diagnosis. As illustrated in FIG. 2, position information of each of the tumor image 101t obtained from the ultrasound image data 204 and the blood vessel image 102v obtained from the photoacoustic image data 202 is provided from the radiogram interpretation image acquisition unit 205 to the reference image acquisition unit 213. The reference image acquisition unit 213 stores each of the photoacoustic image data 214 and the ultrasound image data 215 extracted from the past data storage unit 207 as a previously acquired image.

The reference image acquisition unit 213 generates reference image data 223 to be output to the side-by-side display unit 217 by using the photoacoustic image data 214 and the ultrasound image data 215. The reference image acquisition unit 213 according to the present exemplary embodiment is configured to use the image generation data 216 when generating the reference image 102 superimposing the photoacoustic image 102p and the ultrasound image 102u.

The reference image acquisition unit 213 performs processing for superimposing the photoacoustic image data 214 and the ultrasound image data 215. This means that the reference image acquisition unit 213 generates the reference image 102 superimposing the blood vessel image 102v and the tumor image 102t. When the reference image acquisition unit 213 generates the reference image data 223, the reference image acquisition unit 213 acquires the photoacoustic image data 214, the ultrasound image data 215, and the image generation data 216 used to generate the reference image data 223 from the past data storage unit 207. Previously acquired data stored in the past data storage unit 207 includes the past ultrasound image data 209 and the past photoacoustic image data 208 captured before the radiogram interpretation image 101 is captured.

The reference image acquisition unit 213 can be configured to directly acquire the photoacoustic image data 214 and the ultrasound image data 215 as previously acquired image data from the image database 220 without using the past data storage unit 207. More specifically, the reference image acquisition unit 213 can access the image database 220 storing the photoacoustic image data 214 and the ultrasound image data 215 in an associated way. The reference image acquisition unit 213 according to the present exemplary embodiment acquires a previous image of one or more of the photoacoustic image data 214 or the ultrasound image data 215 from the image database 220 via the past data storage unit 207.

The previously acquired data to be stored in the past data storage unit 207 from the image database 220 can be specified by the radiogram interpreter or can be automatically selected by the past data storage unit 207 based on the radiogram interpretation image data 225 or the radiogram interpretation image 101. As a method for selecting a reference image from the past data storage unit 207 by the reference image acquisition unit 213, there is a method of giving priorities based on the previously acquired image data to candidate images of the reference image by inputting a keyword in the information input unit 212. The candidate images of the reference image are prioritized based on data on correlation with a case stored in the past data storage unit 207 or the image database 220. More specifically, the prioritization is performed based on the photoacoustic image data 208, the ultrasound image data 209, the diagnostic result data 210, and the image generation data 211 accumulated in the past data storage unit 207 and the image database 220, and a search result obtained through a keyword search.

The image database 220 can store an image and image information acquired by a photoacoustic apparatus and an ultrasound apparatus (not illustrated) that are not connected to the medical imaging apparatus 200. More specifically, via the image database 220, the medical imaging apparatus 200 can acquire an image and image information acquired by a photoacoustic apparatus and an ultrasound apparatus (not illustrated) that are not directly connected and store the image and the image information in the past data storage unit 207. In other words, the reference image acquisition unit 213 can display a candidate group of reference images in a predetermined arrangement form based on the related information about, for example, a case specified by a keyword. Based on the related information, the reference image acquisition unit 213 is configured to display candidate groups 103 and 104 of reference images from the image database 220 to enable an operator to specify a reference image. The operator includes a radiogram interpreter, a radiogram interpretation doctor, an ultrasonic imaging operator, and a photoacoustic imaging operator.

In inputting a keyword, the radiogram interpreter can manually input arbitrary related information. Alternatively, information preset from the metadata of the radiogram interpretation image 101 and the image generation parameter, or arbitrary information preset by the radiogram interpreter can be automatically input to the information input unit 212. When the radiogram interpreter manually inputs a keyword, the radiogram interpreter can input an arbitrary character from a keyboard or use a graphical user interface (GUI) for selecting a frequently used keyword from a pull-down menu. The keyword to be input includes the related information about a case related to the radiogram interpretation image 101. The case related to the radiogram interpretation image 101 includes a similar case suspected to be positive for the tumor image 101t included in the radiogram interpretation image 101 and an analogous case to be denied, i.e., an analogous case to be differentiated.

### <Reference Image Superimposition Conditions>

The reference image acquisition unit 213 can generate both a superimposed image and a projected image by using volume data of each of the photoacoustic image data 214 and the ultrasound image data 215.

The reference image 102 superimposing the ultrasound image 102u and the photoacoustic image 102p is generated based on the superimposed image generation parameter specified to the reference image acquisition unit 213 by the radiogram interpreter. The reference image 102 can also be generated based on the image generation data 216. The image generation data 216 includes image generation information of a superimposed image generated at the time of previous image diagnosis, which can be used to generate the reference image 102.

The reference image acquisition unit 213 can generate the reference image data 223 with reference to superimposition conditions used to generate the radiogram interpretation image data 225 by the radiogram interpretation image acquisition unit 205.

Examples of parameters related to superimposition conditions used to generate the reference image data 223 by the reference image acquisition unit 213 are described below. The parameters related to superimposition conditions include an image gradation display parameter, a projection range, a superimposition ratio, and an image pseudo color display setting. The image gradation display parameter includes a window value and a window width value. Both the window value and the window width value is set based on luminance value distribution information within a specified region.

The reference image acquisition unit 213 can generate the reference image data 223 by superimposing, on the photoacoustic image data 214, ROI information about a lesion area generated based on the ultrasound image data 215 instead of the ultrasound image data 215.

Similar to an image of the radiogram interpretation image acquisition unit 205, an image generated by the reference image acquisition unit 213 includes a superimposed image of slice images, a superimposed image of a slice image and a projected image, a superimposed image of projected images, and a superimposed image of a slice image, a projected image, and the ROI information.

If the reference image data stored in the past data storage unit 207 is volume data, the reference image acquisition unit 213 can generate a pseudo moving image (slices of two-dimensional images are sequentially fed) by making a setting of a slice range to be two-dimensionally displayed on the screen. When such a pseudo moving image is used as the reference image 102, loop reproduction is possible. A three-dimensional image of a periphery of the lesion area can be generated by using the previously acquired image data in a volume data format stored in the past data storage unit 207.

If a slice image is acquired from the volume data as the reference image, it is necessary to specify the slice image number to be selected. The slice image can be selected by inputting position information of a target slice image or by checking slice images while feeding slices and selecting position information when a desired image appears.

### <Side-by-Side Display Unit>

As illustrated in FIGS. 1A, 1B, 1C, 1D, and 1E, the side-by-side display unit 217 displays the radiogram interpretation image 101 (based on the radiogram interpretation image data 225) and the reference image 102 (based on the reference image data 223) side by side on the display unit 218. The adjustment value input unit 219 (described below) adjusts image quality of each of the radiogram interpretation image 101 and the reference image 102 displayed side by side by the side-by-side display unit 217.

### <Display Unit>

The display unit 218 is a display device such as a liquid crystal display, an organic light emitting diode (LED), an inorganic LED, a glasses-type display, and a head-mounted display. As illustrated in FIGS. 1A, 1B, 1C, 1D, and 1E, the display unit 218 displays the radiogram interpretation image 101, the reference image 102, diagnostic result data, a part of the image generation data, and a GUI for operating the medical imaging apparatus or the imaging apparatus based on an image signal output from the side-by-side display unit 217. The display unit 218 uses a single display configuration with a single display screen or a multi-display configuration with a plurality of display devices.

### <Adjustment Value Input Unit>

The adjustment value input unit 219 includes an adjustment parameter input unit enabling the radiogram interpreter, while monitoring the screen, to readjust the radiogram interpretation image 101 and the reference image 102 displayed side by side by the side-by-side display unit 217. The adjustment value input unit 219 can adjust the image quality of each of the radiogram interpretation image 101 and the reference image 102 in a separate way or in a linked way.

The adjustment value input unit 219 enables input of setting values. An image quality parameter that can be adjusted by the adjustment value input unit 219 includes luminance, contrast, image quality (gamma curve), an enlargement factor, a display range, a gradation adjustment (based on a window value and a window width value), a projection range, a superimposition ratio, and color mapping selection.

For example, when the superimposition ratio of the radiogram interpretation image 101 is set to 1/2, the superimposition ratio of the reference image 102 can also be set to 1/2. According to the present specification, the superimposition ratio is determined relative to an entire region to be superimposed and is defined as a luminance value of a photoacoustic image divided by a sum of the photoacoustic image and the ultrasound image. A case is described below where, in the superimposed image 101 (102) superimposing the blood vessel image 101v (102v) and the tumor image 101t (102t), the superimposition ratio is changed to emphasize the blood vessel image 101v in the radiogram interpretation image 101. If the superimposition ratios of the reference image 102 and the radiogram interpretation image 101 are changed in a linked way, it becomes possible to compare the reference image 102 with the radiogram interpretation image 101 under the same condition, thereby facilitating radiogram interpretation.

If the radiogram interpretation image 101 is volume data, the projection range of the photoacoustic image 101p in the radiogram interpretation image 101 can be changed from 0.1 mm to 1.0 mm to increase a slab thickness before checking blood vessel connection. When the slab thickness of either one of the radiogram interpretation image 101 and the reference image 102 is changed, by changing the slab thickness of another image in a linked way, it becomes easier to grasp the running state of the blood vessels in both of the blood vessel images 101v and 102v.

### <Flowchart of Processing for Generating Reference Image>

A method for displaying a reference image in which a superimposed image according to the present exemplary embodiment is displayed as the reference image will be described below with reference to the flowchart illustrated in FIG. 3.

### (S100: Acquire Radiogram Interpretation Image)

In step S100, the medical imaging apparatus 200 stores, in the radiogram interpretation image acquisition unit 205, the photoacoustic image data 202 and the ultrasound image data 204 acquired from the photoacoustic apparatus 201 and the ultrasound apparatus 203, respectively. The radiogram interpretation image acquisition unit 205 performs processing for superimposing the photoacoustic image data 202 and the ultrasound image data 204 to acquire the radiogram interpretation image 101 superimposing the photoacoustic image 101p and the ultrasound image 101u. The radiogram interpretation image acquisition unit 205 performs processing for superimposing the photoacoustic image data 202 and the ultrasound image data 204 to generate the radiogram interpretation image data 225, which is output to the side-by-side display unit 217.

### (S101: Input Keyword in Information Input Unit)

The information input unit 212 receives a keyword input. The keyword to be input in this step is a search term used to search for the candidate images 103 and 104 for the reference image 102 correlated with the radiogram interpretation image 101. The keyword to be input can be replaced with a keyword acquired from an inspection order or an electronic medical chart (not illustrated). In other words, the input by the operator and case information included in the electronic medical chart or the inspection order are related information about the case.

### (S102: Perform Search and Prioritize Candidate Images)

Based on the keyword input in step S101, the reference image acquisition unit 213 searches for the photoacoustic image data 214 and the ultrasound image data 215 in the photoacoustic image data 208 and the ultrasound image data 209, respectively, stored in the past data storage unit 207, and stores the searched image data. The reference image acquisition unit 213 also performs prioritization of previously acquired images having high correlation with the radiogram interpretation image 101 by using the keyword input from the diagnostic result data 210 and the image generation data 211.

### (S103: Acquire Reference Image)

By using the reference image candidate with the highest priority in step S102, the reference image acquisition unit 213 performs processing for superimposing the photoacoustic image data 214 and the ultrasound image data 215 to generate a temporary reference image 102 superimposing the photoacoustic image 102p and the ultrasound image 102u. The reference image acquisition unit 213 performs processing for superimposing the photoacoustic image data 214 and the ultrasound image data 215 to generate the reference image data 223, which is output to the side-by-side display unit 217.

The reference image acquisition unit 213 outputs a similar image with the second or lower priority and an analogous disease image to be differentiated to the side-by-side display unit 217 so that these images are displayed on the display unit 218 as the candidate images 103 and 104.

### (S104: Display Images Side by Side and Input Image Diagnostic Result)

The side-by-side display unit 217 displays the radiogram interpretation image 101 and the reference image 102 side by side on the display unit 218. When the radiogram interpreter determines that the reference image 102 is an appropriate image serving as an index of a case of the radiogram interpretation image 101 (YES in step S104), the radiogram interpreter inputs, in the information input unit 212, an instruction notifying that the reference image 102 is an appropriate image. Based on the input instruction notifying that the reference image 102 is an appropriate image from the radiogram interpreter, the reference image acquisition unit 213 receives an instruction to proceed to step S105.

When the radiogram interpreter does not determine that the reference image 102 is an appropriate image (NO in step S104), the radiogram interpreter selects a new reference image 102 from the candidate images 103 and 104. Then, the reference image 102 in the display screen 100 is updated. Based on the input instruction notifying that the reference image 102 is not an appropriate image from the radiogram interpreter, the reference image acquisition unit 213 receives an instruction to return to step S101 and receives a keyword input. The reference image acquisition unit 213 repeats a loop from step S101 to step S104 until the radiogram interpreter inputs an instruction notifying that the reference image 102 is an appropriate image in step S104.

In step S104, the radiogram interpreter can acquire the reference image 102 to be an appropriate image via sub step S107 (subroutine) for performing image adjustment on either the radiogram interpretation image 101 or the reference image 102.

### (S105: Input Diagnostic Result and Update Image Generation Data)

The reference image acquisition unit 213 receives an input of an image diagnostic result determined by the radiogram interpretation of the reference image 102 and the radiogram interpretation image 101 by the radiogram interpreter. The reference image acquisition unit 213 outputs the input image diagnostic result in association with the radiogram interpretation image 101 to the past data storage unit 207, thereby updating the diagnostic result data 210. The reference image acquisition unit 213 also outputs the image generation data such as superimposition conditions associated with the reference image 102 and the radiogram interpretation image 101 to the past data storage unit 207, thereby updating the image generation data 211.

### (S106: Update Image Database)

The past data storage unit 207 outputs the photoacoustic image data 208, the ultrasound image data 209, the diagnostic result data 210, and the image generation data 211 updated in the past data storage unit 207 to the image database 220, thereby updating the data.

### <Reference Form>

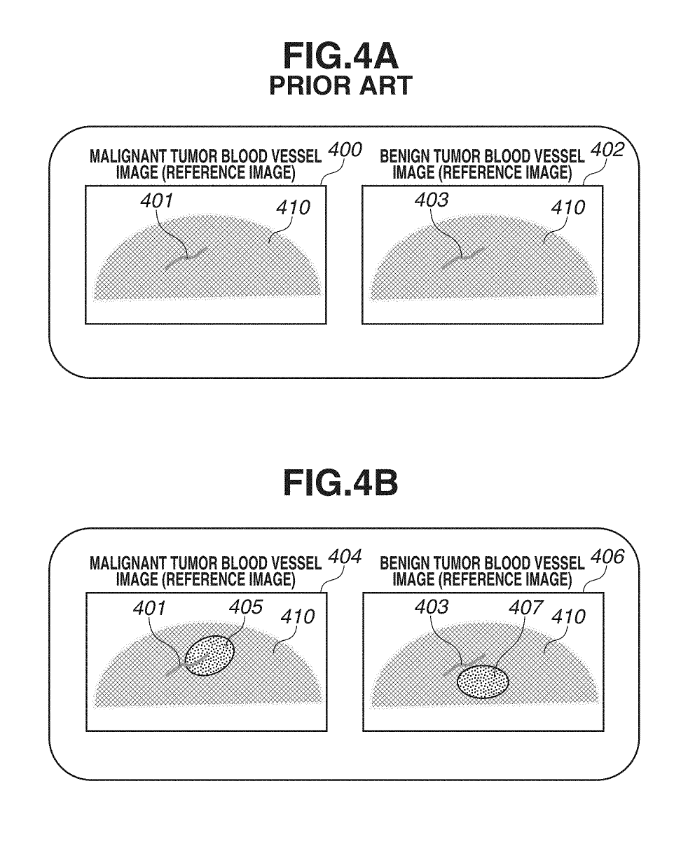

As a reference form in a case where image diagnosis is performed without applying the present disclosure, a case of mastopathy will be specifically described below with reference to FIG. 4A. A photoacoustic image 400 captured so that a tumor that is determined to be malignant in a definite diagnosis is included, includes a blood vessel image 401 and a background image 410 corresponding to background tissues other than the blood vessel. The background image 410 is estimated to include a tumor tissue and a non-tumor tissue including a muscle tissue, an adipose tissue, and a lymph gland. In the background image 410, a tumor region is not separated from the other tissues in photoacoustic imaging, whereby it is estimated that the tumor region exists in an intermediate gradation region having a lower strength than the blood vessel image 401. Since the region corresponding to the tumor and the blood vessel image 401 are not displayed on the same screen, the radiogram interpreter cannot determine the nature of the tumor based on the blood vessel image 401. Thus, the photoacoustic image 400 is not suitable as the reference image.

A photoacoustic image 402 captured so that a tumor that is determined to be benign in a definite diagnosis is included, includes a blood vessel image 403 and a background image 413, similar to the photoacoustic image 400. When the photoacoustic image 400 and the photoacoustic image 402 are compared, the blood vessel image 401 and the blood vessel image 403 look similar because the running states of the blood vessels in the respective tumor regions are unknown. In both of the photoacoustic images 400 and 402, it is difficult to grasp the running state of the blood vessel. Thus, the two images are not appropriate as reference images to be used to determine the nature of the tumor.

More specifically, the photoacoustic images 400 and 402 are photoacoustic images of mastopathy including a malignant tumor and a benign tumor, respectively. The blood vessel images 401 and 403 represented in the photoacoustic images 400 and 402, respectively, have unknown positional relations with the tumor portion. Thus, the radiogram interpreter cannot interpret a difference between the blood vessel images 401 and 403 from the photoacoustic images 400 and 402.

FIG. 4B illustrates an image diagnosis as an example application of the present disclosure. In the present invention, an image superimposing a photoacoustic image and an ultrasound image is used as a reference image.

A reference image 404 is displayed by superimposing the blood vessel image 401 and a malignant tumor image 405, whereby it is easier to grasp characteristics of the running state of the blood vessel related to the malignant tumor based on the positional relation between the malignant tumor and the blood vessel. For example, by superimposing the blood vessel image 401 and the malignant tumor image 405, it is easier to grasp characteristics of the blood vessel of the blood vessel image 401 that approximately perpendicularly penetrates into the malignant tumor 405.

Likewise, the reference image 406 is displayed by superimposing the blood vessel image 403 and the benign tumor image 407, whereby it is easier to grasp characteristics of the running state of the blood vessel related to the benign tumor based on the positional relation between the benign tumor and the blood vessel.

In this way, using a superimposed image of a photoacoustic image and an ultrasound image as a reference image is effective to grasp characteristics relative to a lesion position based on a blood vessel image of the photoacoustic image.

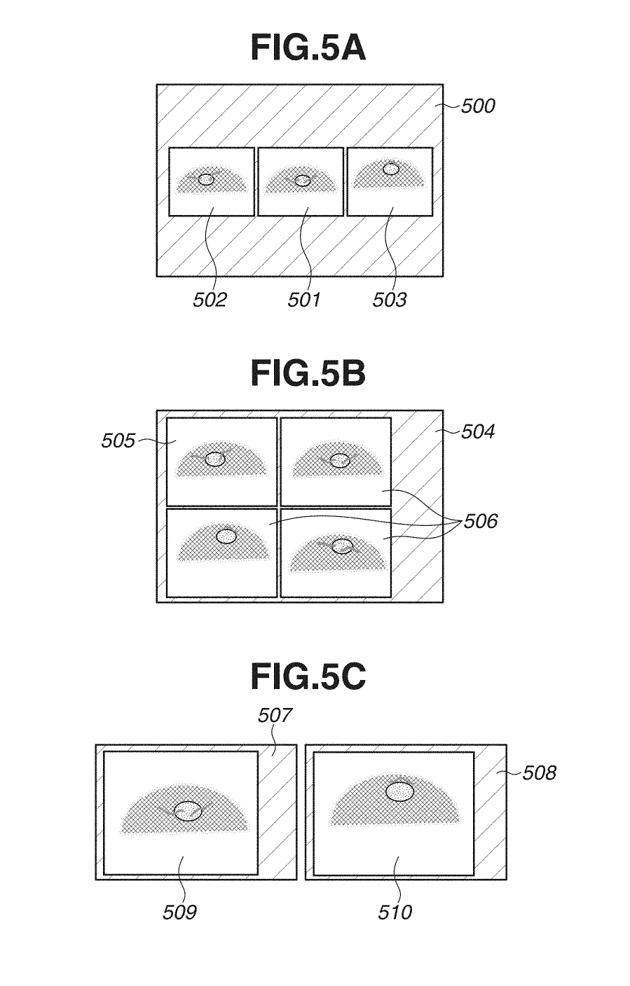

A method for displaying a medical image according to a second exemplary embodiment will be described below with reference to FIGS. 5A to 5C. Examples according to the present exemplary embodiment are different from examples of the first exemplary embodiment in a screen layout of the radiogram interpretation image and the reference image.

In FIG. 5A, a display screen 500 includes a radiogram interpretation image 501 and reference images 502 and 503 linearly arranged in a 1×3 array form so that the reference images 502 and 503 horizontally sandwich the radiogram interpretation image 501. Displaying the plurality of reference images 502 and 503 in this way to compare them with the radiogram interpretation image 501 is also effective. Multifaceted diagnosis can be performed by displaying different types of reference images such as the reference image 502 for an estimated disease and the reference image 503 for a disease to be differentiated. The arrangement of the radiogram interpretation image 501 and the reference images 502 and 503 is to be considered as an example and can be arbitrarily set by the radiogram interpreter.

In FIG. 5B, a display screen 504 includes a radiogram interpretation image 505 and three reference images 506 arranged in a 2×2 matrix form. Such a two-dimensional layout provides an effect of facilitating finding of an influence (correlation) of a specific parameter included in the radiogram interpretation image, such as (no adjustment, adjustment a1, and adjustment a2), (adjustment a, adjustment b, and adjustment a+b), and (case A, case B, and adjustment case A+B).

In FIG. 5C, on display screens 507 and 508, a radiogram interpretation image 509 and a reference image 510 are arranged, respectively, by using a multi-display configuration so that the images are largely displayed.

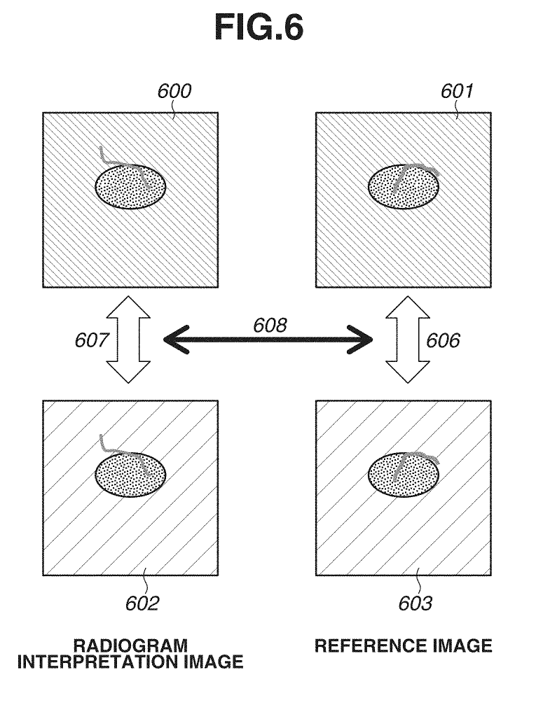

A method for adjusting a radiogram interpretation image and a reference image will be described below with reference to FIG. 6. A third exemplary embodiment is different from the first exemplary embodiment in the display layout when an image adjustment is performed on the radiogram interpretation image and the reference image. FIG. 6 illustrates an example display of a radiogram interpretation image 600, a reference image 601, an adjustment window 602 for the radiogram interpretation image 600, and an adjustment window 603 for the reference image 601 displayed in a 2×2 matrix form.

When the radiogram interpreter performs an image adjustment 606 on the reference image 601 via the adjustment value input unit 219, the adjustment window 603 is updated to an adjusted state. At this time, an image adjustment 607 is performed on the radiogram interpretation image 600 in a link 608 with the image adjustment 606 on the reference image 601.

The adjustment window 602 for the radiogram interpretation image 600 enables the radiogram interpreter to check an adjustment result of the image adjustment 607 performed on the radiogram interpretation image 600 in link with the image adjustment 606 on the reference image 601. Conversely, the image adjustment 606 can be performed on the reference image 601 in the link 608 with the image adjustment 607 on the radiogram interpretation image 600.

As described above, when an image adjustment is performed on either the radiogram interpretation image or the reference image, an image display condition is also changed for another image in link with the image adjustment. More specifically, when an ultrasound image component is increased in the superimposition ratio of the radiogram interpretation image, the superimposition ratio of the reference image is also changed in a linked way. In this case, the change of the superimposition ratio in the linked way can be made on an absolute value basis or on a relative basis.

In the change of the superimposition ratio on the absolute value basis, when the superimposition ratio of the radiogram interpretation image is changed from 50% to 25% and the superimposition ratio of the reference image is 60%, the superimposition ratio thereof is also changed from 60% to 25%. In the superimposition ratio change on the relative basis, when the superimposition ratio of the radiogram interpretation image is halved from 50% to 25% and the superimposition ratio of the reference image is 60%, the superimposition ratio thereof is also changed from 60% to 30%.

While exemplary embodiments have been described, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2017-240948, filed Dec. 15, 2017, which is hereby incorporated by reference herein in its entirety.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

What is claimed is:

1. A medical imaging apparatus, comprising:
a radiogram interpretation image acquisition unit configured to acquire a radiogram interpretation image superimposing an ultrasound image and a photoacoustic image so that imaging angles of view to a subject overlap with each other;
a reference image acquisition unit configured to acquire a reference image to estimate a nature of the radiogram interpretation image; and
a side-by-side display unit configured to display the radiogram interpretation image and the reference image side by side,
wherein the reference image acquisition unit acquires the reference image superimposing a tumor image and a blood vessel image.

2. The medical imaging apparatus according to claim 1,
wherein the reference image acquisition unit accesses an image database storing photoacoustic image data and ultrasound image data in association with each other, and
wherein the reference image acquisition unit acquires the tumor image and the blood vessel image from the image database and superimposes the images to acquire the reference image.

3. The medical imaging apparatus according to claim 2, wherein the reference image acquisition unit acquires the reference image from the image database based on one or more of image information extracted based on the radiogram interpretation image or related information about a case.

4. The medical imaging apparatus according to claim 3, wherein the image information includes parametric information extracted based on the radiogram interpretation image.

5. The medical imaging apparatus according to claim 3, wherein the related information includes an input by an operator, case information included in an electronic medical chart, and an inspection order.

6. The medical imaging apparatus according to claim 5, wherein the reference image acquisition unit displays, based on the related information, a candidate group of reference images enabling the operator to specify the reference image from the image database.

7. The medical imaging apparatus according to claim 6, wherein the reference image acquisition unit arranges and displays, based on the related information, the candidate group in a predetermined array form.

8. The medical imaging apparatus according to claim 2, further comprising a past data storage unit configured to store the photoacoustic image data, the ultrasound image data, and previously acquired photoacoustic image data and ultrasound image data, wherein the past data storage unit is accessibly connected to the image database.

9. The medical imaging apparatus according to claim 8 wherein the reference image acquisition unit acquires, from the past data storage unit, a previously acquired image of one or more of the photoacoustic image data or the ultrasound image data from the image database.

10. The medical imaging apparatus according to claim 1, wherein the tumor image is an ultrasound image captured by an ultrasound apparatus before the radiogram interpretation image is captured.

11. The medical imaging apparatus according to claim 10, wherein the tumor image is associated with a nature of a tumor obtained by a differential diagnosis.

12. The medical imaging apparatus according to claim 11, wherein the image database stores the photoacoustic image, the ultrasound image, and information about a diagnostic result storing a result of the differential diagnosis in association with each other.

13. The medical imaging apparatus according to claim 1, wherein the blood vessel image is a photoacoustic image captured by a photoacoustic apparatus before the radiogram interpretation image is captured.

14. The medical imaging apparatus according to claim 13, wherein the blood vessel image is associated with a nature of a tumor obtained by a differential diagnosis.

15. The medical imaging apparatus according to claim 1, wherein, when a display condition of the reference image or the radiogram interpretation image is changed, a display condition of another image is changed in a linked way.

16. The medical imaging apparatus according to claim 1, wherein, the reference image acquisition unit superimposes the blood vessel image and the tumor image based on a superimposition condition of the photoacoustic image and the ultrasound image in the radiogram interpretation image to acquire the reference image.

17. The medical imaging apparatus according to claim 16, wherein the superimposition condition is determined based on image generation information associated with the radiogram interpretation image.

18. The medical imaging apparatus according to claim 17, wherein the image database stores the photoacoustic image data, the ultrasound image data, and the image generation information in association with each other.

19. A medical imaging apparatus, comprising:
a radiogram interpretation image acquisition unit configured to acquire a radiogram interpretation image superimposing an ultrasound image and a photoacoustic image so that imaging angles of view to a subject overlap with each other;
a reference image acquisition unit configured to acquire a reference image to estimate a nature of the radiogram interpretation image; and
a side-by-side display unit configured to display the radiogram interpretation image and the reference image side by side,
wherein the reference image acquisition unit acquires the reference image superimposing a previously acquired ultrasound image and a previously acquired photoacoustic image captured before the radiogram interpretation image is captured.

20. A method for displaying medical images, comprising:
acquiring a radiogram interpretation image superimposing an ultrasound image and a photoacoustic image so that imaging angles of view to a subject overlap with each other;
acquiring a reference image to estimate a nature of the radiogram interpretation image; and
displaying the radiogram interpretation image and the reference image side by side,
wherein, in acquiring the reference image, the reference image superimposing a tumor image and a blood vessel image is acquired.
